(19) United States
(12) Patent Application Publication
(10) Pub. No.: US 2009/0203086 A1
(43) Pub. Date: Aug. 13, 2009

(54) System and method for improved signal detection in nucleic acid sequencing

(75) Inventors: Yi-Ju Chen, New Haven, CT (US); Jong-Bum Kim, Branford, CT (US); Andrew Daniel Kebbel, Hamden, CT (US); James Matthew Nealis, East Lyme, CT (US); Zhoutao Chen, Carlsbad, CA (US)

Correspondence Address: Ivor R. Elrifi;Mintz, Levin, Cohn, Ferris, Glovsky and Popeo, P.C, 24th Floor, 666 Third Avenue, New York, NY, 10017, US

(73) Assignee: 454 Life Sciences Corporation

(21) Appl. No.: 12/322,284

(22) Filed: Jan. 29, 2009

Related U.S. Application Data
(60) Provisional application No. 61/026,547, filed on Feb. 6, 2008.

(51) Int. Cl. C12P 19/34; B01J 8/00; C12M 1/00; C07H 1/00

(52) U.S. Cl. 435/91.5; 422/188; 435/289.1; 536/55.3

(57) ABSTRACT

An embodiment of a system for reducing crosstalk in a parallel sequencing platform is described that comprises a substrate with a plurality of individual reaction environments that include a species of nucleic acid template, and a plurality of spatially localized reactants, wherein the localized reactants minimize the transmission of reaction products to a neighboring reaction environment due to a relative position of the localized reactants in the reaction environment.

### CROSS-REFERENCE TO RELATED APPLICATIONS

This application is related to and claims priority from U.S. Provisional Patent Application Ser. No. 61/026,547, titled “System and Method for Improved Signal Detection in Nucleic Acid Sequencing”, filed Feb. 6, 2008, which is hereby incorporated by reference herein in its entirety for all purposes.

### FIELD OF THE INVENTION

The present invention relates to the fields of molecular biology and nucleic acid sequencing instrumentation. More specifically, the invention relates to improving signal discretion and detection in nucleic acid sequencing methods.

### BACKGROUND OF THE INVENTION

There have been a number of advancements in the field of Molecular Biology that have enabled the development of many technologies that provide great insight into the nature of biological mechanisms. The power of some of these technologies have made great impacts upon scientific discovery and hold great promise for the future. One particular area that has seen a great amount of advancement is the field of nucleic acid sequencing, where a number of technological advances have been developed enabling a great reduction in the cost of sequencing and substantially increasing the amount of sequence data produced by sequencing many nucleic acid molecules in parallel.

There are a number of “nucleic acid sequencing” technologies known in the art amenable for use with the presently described invention such as, for instance, techniques based upon what are referred to as Sanger type sequencing methods commonly known to those of ordinary skill in the art that employ termination and size separation techniques to identify nucleic acid composition. Other techniques amenable for use with the presently described invention include Sequencing by Hybridization (SBH) or Sequencing by Ligation techniques. Another class of powerful sequencing techniques includes what are referred to as “sequencing-by-synthesis” techniques (SBS). SBS techniques are generally employed for determining the identity or nucleic acid composition of one or more molecules in a nucleic acid sample. SBS techniques provide many desirable advantages over previously employed sequencing techniques. For example, embodiments of SBS are enabled to perform what are referred to as high throughput sequencing that generates a large volume of high quality sequence information at a low cost relative to previous techniques. A further advantage includes the simultaneous generation of sequence information from multiple template molecules in a massively parallel fashion. In other words, multiple nucleic acid molecules derived from one or more samples are simultaneously sequenced in a single process.

Typical embodiments of SBS comprise the stepwise synthesis of strands of polynucleotide molecules each complementary to a strand from a population of substantially identical template nucleic acid molecules. For example, SBS techniques typically operate by adding a single nucleotide (also referred to as a nucleotide or nucleic acid species) to each nascent polynucleotide molecule in the population where the added nucleotide species is complementary to a nucleotide species of a corresponding template molecule at a particular sequence position. The addition of the nucleic acid species to the nascent molecules typically occur in parallel for the population at the same sequence position and are detected using a variety of methods known in the art that include, but are not limited to what are referred to as pyrosequencing or fluorescent detection methods such as fluorescent detection techniques employing reversible or “virtual” terminators (the term virtual terminator as used herein generally refers to terminators substantially slow reaction kinetics where additional steps may be employed to stop the reaction such as the removal of reactants) or energy transfer labels including fluorescent resonant energy transfer dyes (FRET). Typically, the process is iterative until a complete (i.e. all sequence positions of the target nucleic acid molecule are represented) or desired sequence length complementary to the template is synthesized.

In some embodiments of SBS a number of enzymatic reactions take place in order to produce a detectable signal from each incorporated nucleic acid species. In the example of the pyrosequencing SBS method referred to above what may be referred to as an enzymatic cascade is employed, where each enzyme species in the cascade operates to modify or utilize the product from a previous step. For example, as those of ordinary skill in the art understand when each nucleotide species is incorporated into the nascent strand there is a release of an inorganic pyrophosphate (also referred to as PPi) molecule into the reaction environment. The ATP sulfurylase enzyme is present in the reaction environment and converts PPi to ATP, which in turn is catalyzed by the luciferase enzyme to release a photon of light. It will also be appreciated by those of ordinary skill that additional enzymes may be used in the cascade to improve the discretion of signals between exposures to different nucleotides species as well as the overall ability to detect signals.

Further, as described above many embodiments of SBS are enabled to perform sequencing operations in a massively parallel manner. For example, some embodiments of SBS methods are performed using instrumentation that automates one or more steps or operation associated with the preparation and/or sequencing methods. Some instruments employ elements such as plates with wells or other type of microreactor configuration that provide the ability to perform reactions in each of the wells or microreactors simultaneously. Additional examples of SBS techniques as well as systems and methods for massively parallel sequencing are described in U.S. Pat. Nos. 6,274,320; 6,258,568; 6,210,891, 7,211,390; 7,244,559; 7,264,929; 7,335,762; and 7,323,305 each of which is hereby incorporated by reference herein in its entirety for all purposes; and U.S. patent application Ser. No. 11/195,254, which is hereby incorporated by reference herein in its entirety for all purposes.

It will be appreciated that many massively parallel sequencing formats suffer from what are referred to as “crosstalk” effects where a signal or product from one reaction environment is transmitted or transferred to one or more neighboring environments producing an erroneous signal. For example, in embodiments that employ enzymatic reactions to produce a light signal crosstalk can be a chemical product of one or more of the reactions and/or an optical product of light. As reaction environments become increasingly small and compact, such as for instance by decreasing well dimensions of an optical fiber faceplate, crosstalk effects become more pronounced and problematic. In the present example, embodiments of parallel sequencing format may employ a number of enzymatic strategies to control crosstalk effects such as the use of apyrase that degrades unincorporated nucleotide species and ATP, exonuclease that degrades linear nucleic acid molecules, pyrophosphatase (also referred to as PPi-ase) which degrades PPi, or enzymes that inhibit activity of other enzymes. However, such enzymatic strategies should be carefully implemented in order to reduce the crosstalk without negatively affecting the ability to detect the real signals. For instance, it is desirable to both control the products of enzymatic reaction steps to reduce crosstalk contamination while at the same time employing those same products at sufficient levels to detect the real signal from a nucleotide incorporation event.

Therefore, it is significantly advantageous to employ methods to optimize conditions within individual reaction environments using enzymatic strategies and a preferred physical distribution of reactants in order to promote significant advantages in signal discretion and recognition.

### SUMMARY OF THE INVENTION

Embodiments of the invention relate to the determination of the sequence of nucleic acids. More particularly, embodiments of the invention relate to methods and systems for reducing crosstalk error in data obtained during the sequencing of nucleic acids.

An embodiment of a system for reducing crosstalk in a parallel sequencing platform is described that comprises a substrate with a plurality of individual reaction environments that include a species of nucleic acid template, and a plurality of spatially localized reactants, wherein the localized reactants minimize the transmission of reaction products to a neighboring reaction environment due to a relative position of the localized reactants in the reaction environment.

Further, a method for reducing crosstalk in a parallel sequencing platform is described that comprises providing a substrate comprising a plurality of individual reaction environments where the reaction environments include a species of nucleic acid template, and a plurality of spatially localized reactants; and exposing the reaction environments to a nucleotide species to produce a detectable signal within one or more of the reaction environments, wherein the localized reactants within the reaction environments minimize the transmission of reaction products to a neighboring reaction environment.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument comprising optic and fluidic subsystems for processing a reaction substrate under computer control;

FIG. 2 is a functional block diagram of one embodiment of the optic and fluidic subsystems of FIG. 1 for processing the reaction substrate;

FIG. 3 is a simplified graphical representation of one embodiment of a plurality of reaction environments exhibiting optical and chemical crosstalk effects;

FIGS. 4A and 4B are simplified graphical representations of embodiments of reactant distribution within a reaction environment; and

FIG. 5 is a simplified graphical representation of one embodiment of a comparison of the plurality of reaction environments of FIG. 3 illustrating the effects produced by employing intra-well reactant distribution strategies.

### DETAILED DESCRIPTION OF THE INVENTION

As will be described in greater detail below, embodiments of the presently described invention include systems and methods for optimized signal discretion and detection.

a. General

The terms “flowgram” and “pyrogram” may be used interchangeably herein and generally refer to a graphical representation of sequence data generated by SBS methods.

Further, the term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a serial or iterative cycle of addition of solution to an environment comprising a template nucleic acid molecule, where the solution may include a nucleotide species for addition to a nascent molecule or other reagent such as buffers or enzymes that may be employed to reduce carryover or noise effects from previous flow cycles of nucleotide species.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment”, or “TF” as used herein generally refers to a nucleic acid element of known sequence composition that may be employed for quality control, calibration, or other related purposes.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension”, as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products such as those produced from Polymerase Chain Reaction or Ligase Chain Reaction techniques.

The term “key element” as used herein generally refers to a nucleic acid sequence element (typically of about 4 sequence positions, i.e. TGAC or other combination of nucleotide species) associated with a template nucleic acid molecule in a known location (i.e. typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition in a reaction well, where the accuracy of the sequence derived from keypass test sequence is compared to the known sequence composition and used to measure of the accuracy of the sequencing and for quality control. In typical embodiments a proportion of the total number of wells in a sequencing run will be keypass wells which may in some embodiments be regionally distributed or specific.

The term “blunt end” or “blunt ended” as used herein generally refers to a linear double stranded nucleic acid molecule having an end that terminates with a pair of complementary nucleotide base species, where a pair of blunt ends are always compatible for ligation to each other.

The term “bead” or “bead substrate” as used herein generally refers to a any type of bead of any convenient size and fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

Some exemplary embodiments of systems and methods associated with sample preparation and processing, generation of sequence data, and analysis of sequence data are generally described below, some or all of which are amenable for use with embodiments of the presently described invention. In particular the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample must be prepared and processed from its raw form into template molecules amenable for high throughput sequencing. The processing methods may vary from application to application resulting in template molecules comprising various characteristics. For example, in some embodiments of high throughput sequencing it is preferable to generate template molecules with a sequence or read length that is at least the length a particular sequencing method can accurately produce sequence data for. In the present example, the length may include a range of about 25-30 base pairs, about 50-100 base pairs, about 200-300 base pairs, or about 350-500 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will however, be appreciated that other methods of fragmentation such as digestion using restriction endonucleases may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements, unique identifiers that encode various associations such as with a sample of origin or patient, or other functional element. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In the present example, two sets of priming sequence regions (hereafter referred to as priming sequence A, and priming sequence B) may be employed for strand selection where only single strands having one copy of priming sequence A and one copy of priming sequence B is selected and included as the prepared sample. The same priming sequence regions may be employed in methods for amplification and immobilization where, for instance priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. Provisional Patent Application Ser. No. 61/031,779, titled “System and Method for Improved Processing of Nucleic Acids for Production of Sequencable Libraries”, filed Feb. 27, 2008; and U.S. patent application Ser. No. 12/156,242, titled “System and Method for Identification of Individual Samples from a Multiplex Mixture”, filed May 29, 2008, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid such as a water based fluid suspended or dispersed in what may be referred to as a discontinuous phase within another fluid in what may be referred to as a continuous phase such as an oil based fluid. Further, some emulsion embodiments may employ surfactants that act to stabilize the emulsion that may be particularly useful for specific processing methods such as PCR. Some embodiments of surfactant may include non-ionic surfactants such as sorbitan monooleate (also referred to as Span™ 80), polyoxyethylenesorbitsan monooleate (also referred to as Tween M 80), or in some preferred embodiments dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template or other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. patent application Ser. Nos. 10/861,930; 10/866,392; 10/767,899; 11/045,678 each of which are hereby incorporated by reference herein in its entirety for all purposes.

Also, an exemplary embodiment for generating target specific amplicons for sequencing is described that includes using sets of nucleic acid primers to amplify a selected target region or regions from a sample comprising the target nucleic acid. Further, the sample may include a population of nucleic acid molecules that are known or suspected to contain sequence variants and the primers may be employed to amplify and provide insight into the distribution of sequence variants in the sample.

For example a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (amplicons) is subsequently further amplified individually in separate reaction vessels such as an emulsion based vessel described above. The resulting amplicons (referred to herein as second amplicons), each derived from one member of the first population of amplicons, are sequenced and the collection of sequences, from different emulsion PCR amplicons, are used to determine an allelic frequency.

Some advantages of the described target specific amplification and sequencing methods include a higher level of sensitivity than previously achieved. Further, embodiments that employ high throughput sequencing instrumentation such as for instance embodiments that employ what is referred to as a PicoTiterPlate® array (also sometimes referred to as a PTP® plate or array) of wells provided by 454 Life Sciences Corporation, the described methods can be employed to sequence over 100,000 or over 300,000 different copies of an allele per run or experiment. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005, which is hereby incorporated by reference herein in its entirety for all purposes.

Further, embodiments of sequencing may include Sanger type techniques, techniques generally referred to as Sequencing by Hybridization (SBH) or Sequencing by Incorporation (SBI) that may include what is referred to as polony sequencing techniques; nanopore, waveguide and other single molecule detection techniques; or reversible terminator techniques. As described above a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase enzyme as described in U.S. patent application Ser. No. 12/215,455, titled “System and Method for Adaptive Reagent Control in Nucleic Acid Sequencing”, filed Jun. 27, 2008, which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 150 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. patent application Ser. No. 11/448,462, titled “Paired end sequencing”, filed Jun. 6, 2006, and in U.S. Provisional Patent Application Ser. No. 61/026,319, titled “Paired end sequencing”, filed Feb. 5, 2008, each of which is hereby incorporated by reference herein in its entirety for all purposes.

Some examples of SBS apparatus may implement some or all of the methods described above and may include one or more of a detection device such as a charge coupled device (i.e. CCD camera) or a confocal type architecture, a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include what is referred to as a PTP® array, as described above, formed from a fiber optics faceplate that is acid-etched to yield hundreds of thousands or more of very small wells each enabled to hold a population of substantially identical template molecules (i.e. some preferred embodiments comprise about 3.3 million wells on a 70×75 mm PTP® array at a 35 μm well to well pitch). In some embodiments, each population of substantially identical template molecule may be disposed upon a solid substrate such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. patent application Ser. No. 11/215,458, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. No. 7,323,305 and U.S. patent application Ser. No. 11/195,254 both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. patent application Ser. No. 11/045,678, titled “Nucleic acid amplification with continuous flow emulsion”, filed Jan. 28, 2005, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. Computers typically include known components such as a processor, an operating system, system memory, memory storage devices, input-output controllers, input-output devices, and display devices. It will be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Centrino®, Core™ 2, Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP or Windows Vista®) from the Microsoft Corporation; the Mac OS X operating system from Apple Computer Corp. (such as Mac OS X v10.5 “Leopard” or “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example some commonly employed web browsers include Microsoft® Internet Explorer 7 available from Microsoft Corporation, Mozilla Firefox® 2 from the Mozilla Corporation, Safari 1.2 from Apple Computer Corp., or other type of web browser currently known in the art or to be developed in the future. Also, in the same or other embodiments an internet client may include, or could be an element of, specialized software applications enabled to access remote information via a network such as a data processing application for SBS applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

b. Embodiments of the Presently Described Invention

It will be appreciated that certain “crosstalk” effects may be experienced between closely spaced reaction environments which are detrimental to the ability to effectively detect and discriminate signals generated from specific reactions environments. The term “crosstalk” as used herein generally refers to unwanted detectable signals caused by transference of chemical or optical products between reaction environments that negatively affect the discretion of real signals from their respective reaction environments. The effects of crosstalk are especially important in embodiments that employ arrays of closely spaced wells such as the PTP® plate type fiber optic faceplate arrays described above. In particular, it is highly desirable in most embodiments to reduce inter-well crosstalk of optical and chemical signals to minimal levels so that the signals from each well are true to the reactions occurring within, thereby enabling clear signal recognition and accurate detection of light photons released from each reaction.

For example, in cases where chemical crosstalk is significant and has not been corrected it is necessary to reduce the number of wells on a reaction plate (i.e. PTP® plate) that contain active reactants so that there is a substantially lower number of active wells that are immediate neighbors to each other (i.e. within a 1-2 well distance in all directions on a planar 3 coordinate system). It will be appreciated that substantially reducing the number of active wells also substantially reduces overall throughput of the system. Scaling up the sequencing throughput and efficiency of such systems using reaction plate substrates can be enabled by reducing the effects of crosstalk allowing use of greater proportion of the reaction wells (i.e. increase well loading density) and further allows for an increase in total well density of the array (i.e. via a reduction in well to well pitch, such as a pitch of about 35 μm or less). The term “well loading density” as used herein generally refers to a measure of the number of wells on the reaction substrate area that are loaded with reactants capable of producing a detectable signal in response to the appropriate stimulus.

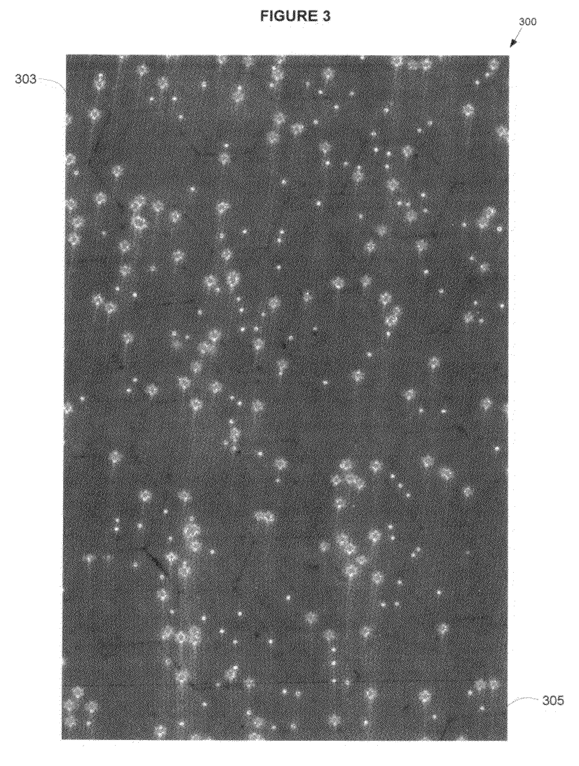

FIG. 3 illustrates image 300 of a well array substrate with reactants distributed at low density that demonstrates the effects of optical and chemical cross talk from individual reaction environments. Streak 303 is a chemical crosstalk effect produced when reactants and/or products escape the reaction environments and follow the direction of fluid flow in a flow cell. For instance, a reaction product such as PPi may escape from the reaction environment through the top of the well in which it was produced and diffuse into a common fluid environment that fluidically interconnects a plurality of the well environments. Typically, the diffusion of reactants or products follows the direction of fluid flow that exists in the common fluid environment however it will be appreciated that the diffusion may also disperse in any direction in fluidic contact, where the diffused reactants or products may enter one or more neighboring well environments and initiate or contribute to a signal cascade of reactions. For example, the diffused molecules typically include the necessary reactants to produce a light signal in response to the PPi. This light product creates a false positive or contaminated signal for an incorporation event in the neighboring well that can be difficult to identify as an experimental artifact. In the present example, a “false positive” signal generally refers to a detectable signal generated in the absence of a nucleic acid template (i.e. a DNA bead) and a “contaminated” signal generally refers to a detectable signal generated in the presence of a nucleic acid template but without the occurrence of an incorporation event of a nucleotide species.

Similarly, halo 305 is an illustrative example of an optical crosstalk effect produced when light reaction product escapes from the reaction environment into neighboring reaction environments. Typically, such transmission occurs laterally through the sidewall surfaces of the reaction wells and into one or more neighboring wells, which again creates a false positive or contaminated signal.

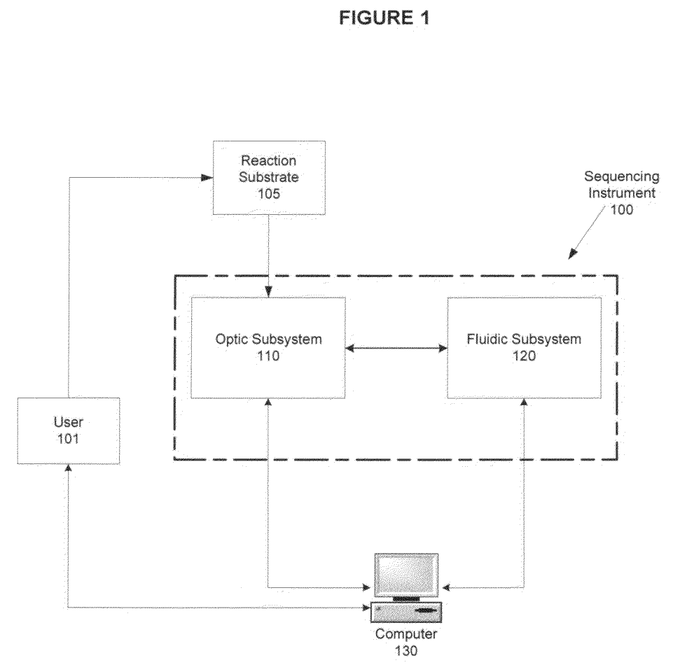

In the described embodiments, one or more instrument elements are employed that automate one or more process steps for introducing reactants, including enzymes, as well as for the steps of measuring and adjusting. For example, embodiments of a sequencing method may be executed using instrumentation and control software to automate and carry out some or all process steps. FIG. 1 provides an illustrative example of sequencing instrument 100 that comprises optic subsystem 110 and fluidic subsystem 120. Embodiments of sequencing instrument 100 employed to execute sequencing processes may include various fluidic components in fluidic subsystem 120, various optical components in optic subsystem 110, and one or more computer components such as computer 130 that may for instance execute system software or firmware that provides instructional control of one or more of the components. In the present example, sequencing instrument 100 and/or computer 130 may include some or all of the components and characteristics of the embodiments generally described above.

Embodiments of fluidic subsystem 120 may include various components such as fluid reservoir A 201, fluid reservoir B 203, fluid reservoir C 205, fluid reservoir D 207, and fluid reservoir E 209 that each may hold a volume of reagent or fluid usable in a sequencing reaction procedure. For example, reservoirs 201-209 may include bottles, flasks, tubes, cuvettes, or other fluid tight receptacle that hold volumes of reagents such as individual nucleotide species (i.e. A, C, G, T, or U); specific enzymes such as apyrase, sulfurylase, luciferase, PPi-ase or other enzyme; test or calibration fluids that may include ATP or PPi; substrates such as adenosine-5′-phosphosulfate (also referred to as APS) or luciferin; enzyme enhancers/activators or inhibitors; wash solutions that may include water and/or dilutions of a bleach, iodine, or other disinfecting solution; or other fluid useable in the sequencing process or for preparation thereof. Embodiments of fluidic subsystem 120 may also include one or more waste reservoirs 240 for recapture and storage of used or spent fluids that are undesirable for re-use. It will be appreciated that multiple embodiments of reservoir 240 may be employed for fluids that are incompatible or dangerous to combine or generally preferable to keep separated.

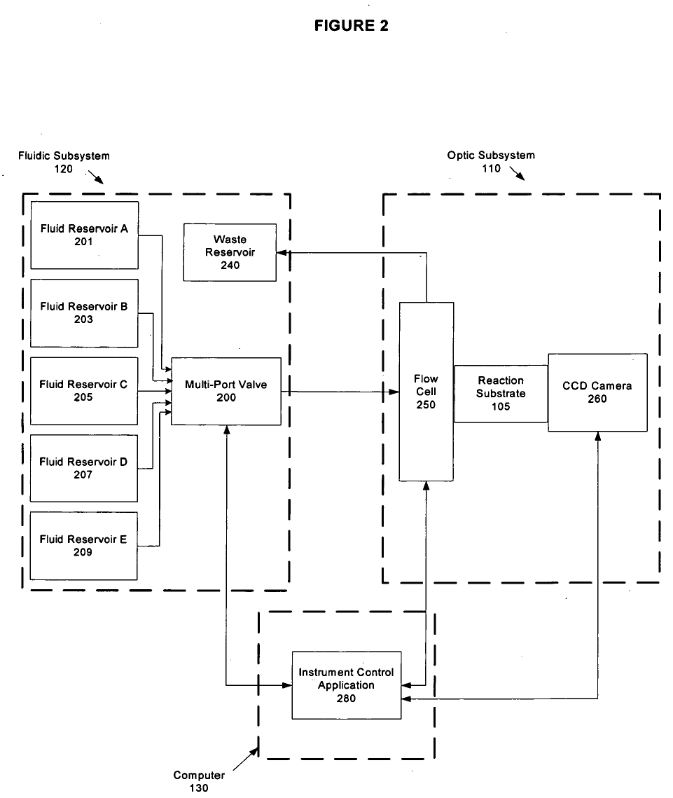

Further each of reservoirs 201-209 may be in fluid communication with multi-port valve 200. In the example of FIG. 2, multi-port valve 200 is enabled to open and close to selectively allow specified volumes of fluid to move from reservoirs 201-209 to flow cell 250. Further, multi-port valve 200 is enabled to adjust rates of flow from one or more of reservoirs 201-209 simultaneously. In the present example, the adjustable flow rate or timing of opening and closing permits for an accurate dilution of reagents. For instance, one possible method for controlling a 10× dilution of a reagent may be achieved by opening a flow from one of reservoirs 201-209 for 1/10^{th} of the total time that reagents are allowed to flow through flow cell 250 where valve 200 may be “pulsed” at intervals (i.e. open vs. closed intervals) to provide better homogeneity of the dilution. Some alternative embodiments may also include controlling a flow rate through valve 200 by modulating the degree that valve 200 is opened. In other words, valve 200 may be opened through a range of a small partial opening to completely open where the rate of flow is dependent upon the degree of opening. It will be appreciated that it may be desirable that the concentrations of one or more reagents in reservoirs 201-209 are exactly known and higher, and in some case substantially higher, than what is desirable for a sequencing process, thus allowing for dilution and easy management of final concentration.

Those of ordinary skill in the related art will also appreciate that fluidic subsystem 120 is exemplary and other components may be included in a typical fluidic subsystem. For instance, some embodiments may include sensors enabled to detect that the correct or expected fluid is present in reservoirs 201-209 or flowing through valve 200. Sensors may include a combination of one or more sensors such as conductivity sensors, optical sensors (i.e. optical density), or acoustic sensors (i.e. ultrasonic density). Some typical fluidic components in subsystem embodiments may also include valves, tubing, pumps (i.e. peristaltic pumps), heating/cooling elements (i.e. heat sink), or other elements commonly employed in the art.

Also illustrated in FIG. 2 are components associated with optic subsystem 110 that include flow cell 250, reaction substrate 105, and CCD camera 260. For example, flow cell 250 is in fluid communication with a first surface of reaction substrate 105 that includes the wells of a PTP® plate housing populations of substantially identical template molecules. Thus the fluid introduced into flow cell 250 is contacted with substrate 105 and the template molecules. Also, in some embodiments what is referred to as a “convective” flow may be established within flow cell 250 for efficient introduction and removal of the reagents from substrate 105. Examples of convective flow in sequencing embodiments and its advantages are described in U.S. patent application Ser. Nos. 10/191,438; 11/016,942; 11/217,194, each of which are hereby incorporated by reference herein in its entirety for all purposes. Additionally, as described above CCD camera 260 is in optical communication with a second surface of reaction substrate 105 so that light generated from sequencing reactions in the wells is transmitted through the bottom surface of the wells to CCD camera 260.

Embodiments of the presently described invention are generally intended to reduce crosstalk effects by effectively controlling the spatial generation and distribution of reaction products within each reaction environment. As described in U.S. patent application Ser. No. 12/215,455, incorporated by reference above, one method for controlling crosstalk effects in a pyrosequencing based strategy over a wide area (i.e. outside of the individual reaction environments) is to wash a reaction vessel or substrate area with what is referred to as “apyrase”. Those of ordinary skill in the related art will appreciate that apyrase is an enzyme that has a number of qualities that include the degradation of nucleoside triphosphates, diphosphates (i.e such as ATP, and PPi (pyrophosphate)). The use of apyrase in SBS embodiments substantially improves the removal of excess and unwanted nucleotide species, reagents, and reaction products over simply washing alone. For example, apyrase may be “washed” or “flowed” over a surface of a solid support comprising one or more reaction areas at the end of each reaction cycle so as to facilitate the degradation of any remaining, non-incorporated nucleotide species molecules within the sequencing reaction mixture. Apyrase may further be employed to degrade ATP generated in a previous cycle and hence “turns off” light generated from the reaction in the previous cycle.

The next reaction cycle with a different nucleotide species may be initiated after a brief washing step that removes remaining apyrase and reaction products. In some embodiments, the apyrase may be bound to the solid or mobile solid support. Additional examples of apyrase use and the advantages conferred by such use are described in U.S. patent application Ser. No. 10/767,779, titled “Methods of amplifying and sequencing nucleic acids”, filed Jan. 28, 2004, which is hereby incorporated by reference herein in its entirety for all purposes.

It will also be appreciated that pyrophosphatase (also referred to as PPiase) may be flowed through reaction environments in the same manner as described above for apyrase at the end of a nucleotide species flow cycle to degrade any unreacted or excess PPi. Those or ordinary skill in the art will appreciate that PPiase acts to catalyze the hydrolysis of diphosphate bonds and degrades nucleoside triphosphates and diphosphates. It will be appreciated that the flow of PPiase may be used independently or combined with the use substrate bound PPiase or other reagents such as the apyrase described above.

In the presently described embodiments it has been found that variation in signal output and crosstalk may be affected, at least in part, by the spatial arrangement of reactants within each reaction environment. Spatial differences in the intra-well distribution of reactants typically result in what are referred to as “layering effects”. The term layering effects as used herein generally refer to differences in localized distribution of substrates, reagents, targets, etc. that may be the result of processes used to distribute the subject material in the reaction environment. The result may include localized variance in the concentration of one or more of the enzymes relative to each other or to populations of template molecules or some combination thereof.

In particular, the spatial distribution of reactants within each reaction environment can be controlled to optimize the reaction efficiencies within each environment while reducing effects upon neighboring reaction environments. This is especially important where the reaction environments are at least partially “open” sharing a common interface or environment. For example, embodiments that employ the PTP® type well structures comprise sidewall surfaces and bottom surfaces defining the boundaries of the reaction environments of each well, however the top of each well is open and in fluid communication with a flow cell effectively linking the reaction environments of the wells to some degree. The spatial distribution of reactants within the reaction environment of each well may be additionally enhanced by the immobilization of some or all of the reactants to sequester them to their preferred spatial location.

In some embodiments of the presently described invention, it is highly desirable to sequester one or more reactant species to preferred regions within the reaction environment. Typically, sequestration is achieved by immobilizing a reactant species upon one or more solid phase substrates, such as beads, and distributing the sequestered reactants in a controlled manner in order to regulate the preferred spatial distribution of each reactant species. For example, it may be highly desirable to distribute the reactant species according to a preferred depth in a well in which a reaction step is desired or expected to take place. It may further be desirable in some contexts to employ additional solid phase substrates that are free of reactants or have specialized functionalization in certain spatial positions in order to occupy space and serve to hold other reactant containing substrates that may be present at a low density in a certain position. In the present example, the low density substrate may include what is referred to as a “DNA” bead comprising a sequestered population of nucleic acid template molecules to be sequenced. It is desirable that the DNA is located in a preferred position within the well, typically some distance away from the top and bottom regions of the well, where non-reactant “packing” beads may be employed to occupy space within the well in order to maintain the preferred position of the DNA bead. In some embodiments it is advantageous that the diameter of the packing bead is substantially smaller than the diameter of the DNA bead to enhance the ability of the beads to compact within the well. It may be further advantageous in some embodiments to exert force on the packing/DNA bead combination to enhance compaction of the beads making them more resistant to alteration of the positional arrangement. One such type of physical force may include centrifugal force that may be accomplished by placing one or more substrates into a centrifuge device and spun for a period of time. Also, for embodiments of reaction substrate coated with an opaque material such as a metal that may be further include an SiO₂ coating (i.e. as described in U.S. patent application Ser. No. 11/215,458 incorporated by reference above), spinning can aid in efficiency of the deposition and packing of bead substrates into the wells.

One class of sequestered reactant may include what are referred to as “degrading” enzymes such as the PPi-ase or apyrase enzymes described above. In preferred embodiments, it is desirable to include a layer of sequestered degrading enzymes at a very low depth within a reaction well (i.e. as a top layer sometimes referred to as a “cap” or “capping” layer, or near the top layer) where the sequestered degrading enzymes do not allow their target reaction products to escape from the reaction environment intact. Thus, the layer acts as a barrier against the reaction products leaving the reaction environment creating crosstalk effects in neighboring reaction environments. Additionally, such a layer of degrading enzyme acts as a barrier against reaction products produced in neighboring reaction environments from entering the reaction environment with the degrading layer.

Another class of sequestered reactant may include what are referred to as “reagent” enzymes such as sulfurylase or luciferase enzymes that are involved in the signal cascade of reactions described above. In preferred embodiments the sequestered reagent substrates are positionally located in the vicinity where the expected reactant or product generated from a previous step will be present for the sequestered reagent to react with. Further, the sequestered reagents are placed in a preferred position for the next reaction product to be utilized. For example, in some embodiments it is advantageous to positionally locate reagent substrates comprising sequestered sulfurylase and/or luciferase at or near the bottom surface of the well. Thus, when the luciferase enzyme reacts with the ATP product light is generated within the bottom region of the well and transmitted more directly through the bottom surface of the well, rather than at a region closer to the top of the well. Thus, generating the light signal near the bottom surface of the well effectively reduces the amount of optical crosstalk of the light to neighboring wells thus promoting improved signal discretion characteristics.

A third class of sequestered reactant may include catalysts or substrates for a reaction such as one or more enzymatic reactions employed in some sequencing technologies. For example, sequestered substrates may include APS employed by sulfurylase to generate an ATP product or luciferin employed by luciferase to generate a light product. In some embodiments it may be preferred to implement the introduction of catalysts or substrates incorporated into a flow of nucleotide species, however it may also be desirable in some embodiments to sequester the substrates or catalysts in the reaction environment.

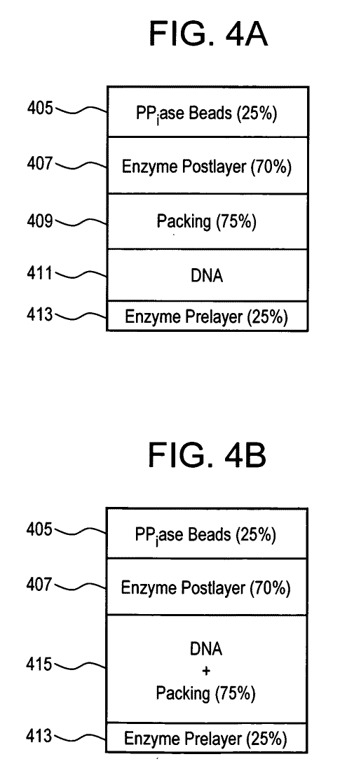

FIGS. 4A and 4B provide illustrative examples of how reactants may be spatially distributed within each reaction environment. For instance, cap layer 405 may comprise a layer of beads with immobilized populations of PPi-ase or another molecule in the class of degrading enzyme disposed thereon as described above. In the described embodiments, the dimension of the beads in layer 405 may include a diameter of about 3 μm. In the present example, chemical cross-talk between reaction environments such as wells limit the operating density of useable wells, which can be remedied via the immobilized PPi-ase cap layer 405 that acts to remove 90%-99% of the pyrophosphate that diffuses in and out of the well reactors and further removes 90%-99% of the background noise in the reagents due to impurities or pyrophosphate contamination in the reagents.

Further, FIGS. 4A and 4B illustrates enzyme prelayer 413 and enzyme postlayer 407 that each comprise beads having immobilized populations of sulfurylase and luciferase enzymes disposed thereon. In the described embodiments, the dimension of the beads in layers 407 and 413 may include a diameter of about 3 μm. Alternatively layers 407 or 413 may include subpopulations of beads where subpopulation includes a single enzyme species disposed thereon such as a first subpopulation comprising sulfurylase and a second subpopulation comprising luciferase. In the presently described embodiment, both layers 407 and 413 include the same proportion of enzyme content, however it will be appreciated that the proportion of enzymes could differ by layer. It may further be advantageous in some embodiments that enzyme layers 407 and 413 include a single enzyme species, where for instance layer 407 may include sulfurylase and layer 413 may include luciferase.

In addition, FIG. 4A illustrates an embodiment comprising packing layer 409 and DNA layer 411. In some embodiments packing layer 409 comprises a layer of avidin or streptavidin coated beads having a small dimension in comparison to the other described bead layers (the dimension of the beads in layer 409 may include a diameter of about 1 μm), particularly DNA layer 411 (the dimension of the bead or beads in layer 411 may include a diameter of about 20 μm). DNA layer 411, in most embodiments, comprises a single bead comprising an immobilized population of substantially identical nucleic acid molecules to be sequenced. The benefit of packing layer 409 includes immobilizing DNA layer 411 in order to maintain the integrity of the spatial distribution.

FIG. 4B illustrates another possible embodiment that combines packing layer 409 and DNA layer 411 into a single commingled layer 415. In some embodiments, the beads of combined layer 411 are functionalized with a coating of avidin that interacts with polymerase molecules used in sequencing methods. The avidin interaction acts to effectively sequester the polymerase molecules in close proximity to the DNA beads with the nucleic acid molecules to be sequenced thereby promoting the interaction between the nucleic acid and the polymerase molecules. For example the functionalized beads effectively trap the relatively large polymerase molecules limiting the intra and inter well diffusion of the polymerase.

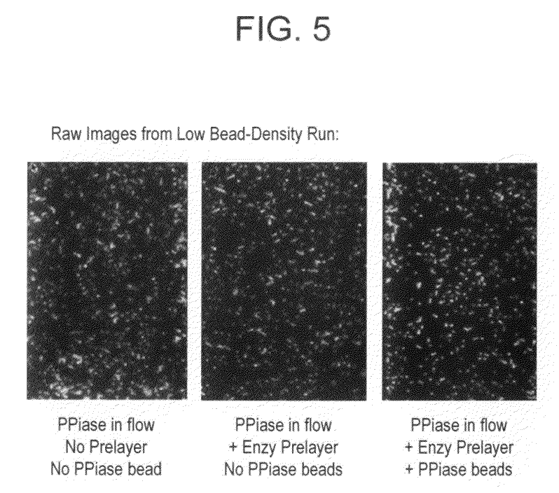

FIG. 5 provides an illustrative example of a comparison of the differences in images produced using the different reactant strategies described above where there is an improvement in the discretion of signal from individual reaction environments as the strategies described herein are employed. The left image illustrates the detected signals when no enzyme prelayer and no PPiase beads are employed, where there is a significant level of noise in the detected signal that causes difficulty in signal discretion. The center image illustrates the detected signals when an enzyme prelayer is employed but no PPiase beads are employed, where the noise in the image is reduced in comparison to the left image, however enough noise remains where signal discretion is negatively impacted particularly at high well densities. The right image illustrates the detected signals when both an enzyme prelayer and PPiase beads are employed, where there is a reduction noise from both the left and center images and there is an improvement in signal discretion.

### Example 1

### Comparison of Performance with and without PPiase Beads

A comparison was performed between wells in different regions of a PTP to test whether PPiase beads on the top layer improved sequencing performance at two different loading densities.

TABLE 1

| Region | 1 | 2 | 3 | 4 |
|---|---|---|---|---|
| Layer 1 Enzyme beads | ¼x | ¼x | ¼x | ¼x |
| Layer 2 DNA beads | 260K | 260K | 800K | 800K |
| | lib1 + | lib1 + | lib2 + | lib2 + |
| | 6K BHTF | 6K BHTF | 6K BHTF | 6K BHTF |
| Layer 3 Packing beads | 1x | 1x | 1x | 1x |
| Layer 4 Enzyme beads | 1x | 1x | 1x | 1x |
| Layer 5 PPiase beads | — | ¼x | ¼x | — |

Lib1: un-enriched
Lib2: enriched

Table 1 illustrates the well layering strategy for regions 1-4, where regions 1-2 included a “low” well loading density of about 5% and regions 3-4 include a “high” well loading density of about 65%. Regions 2 and 3 included a top layer of PPiase beads whereas regions 1 and 4 did not include any PPiase beads. Also, the nucleic acid library used in regions 1 and 2 was different from the nucleic acid library used in regions 3 and 4. Additionally, “x” refers to the standard concentration of beads per ml of solution where x is equal to a concentration in the range of about 5.65−6.58×10⁸ beads/ml.

TABLE 2
Quality Filter

| | | | | | % |
|---|---|---|---|---|---|
| | HQ | HQ | AVG | | HQ/ |
| Region | Reads | Bases | Length | Keypass | KeyPass |
| 1: −PPiase | 3,926 | 1,625,550 | 413.57 | 7112 | 56.38% |
| 2: +PPiase | 5,892 | 2,480,871 | 421.24 | 8080 | 73.56% |
| 3: +PPiase | 247,934 | 105,938,477 | 427.29 | 408,151 | 60.75% |
| 4: −PPiase | 171,859 | 69,133,139 | 401.32 | 448,367 | 38.33% |

TABLE 3
Mapping E. coli

| | | | | | Error@ | Avg |
|---|---|---|---|---|---|---|
| | | | Map | Overall | 400 | Map |
| Region | KeyPass | HQ/KP | bases | Error | bases | Length |
| 1: −PPiase | 7112 | 56.38% | 1.55 M | 1.82% | 1.01% | 402 |
| 2: +PPiase | 8080 | 73.56% | 2.35 M | 1.80% | 0.96% | 408 |
| 3: +PPiase | 408,151 | 60.75% | 99.50 M | 1.88% | 0.93% | 409 |
| 4: −PPiase | 448,367 | 38.33% | 65.27 M | 1.92% | 1.11% | 388 |

Tables 2 and 3 illustrate sequencing results obtained from the regions described in table 1. Both regions 2 and 3 show improved sequencing performance over regions 1 and 4 as illustrated by the High Quality KeyPass measure in addition to other metrics (also denoted as HQ/KP). The High Quality KeyPass numbers show a higher proportion of high quality sequence reads of KeyPass sequence elements in region 2 than in region 1; and similarly there is a higher proportion of high quality sequence reads of KeyPass sequence elements in region 3 than in region 4.

### Example 2

### Performance of Enzyme Pre-Layer

A comparison of sequencing performance was conducted between wells having a layer of enzyme beads adjacent to the bottom surface to wells without such a layer of enzyme beads.

TABLE 4

| | | | Matching key pass | | |
|---|---|---|---|---|---|
| | Sig per | | reads over 200 bp | | |
| Type | base | KeyPass | 100% | 98% | 95% |
| With Prelayer | 574.0 | 155992 | 47.94% | 86.30% | 93.28% |
| No Prelayer | 491.0 | 147741 | 18.08% | 58.57% | 76.93% |

Table 4 illustrates that there is a greater degree of sequencing accuracy in the proportion of KeyPass wells comprising the layer of enzyme beads at the bottom of the well. For instance, the sequences derived from the KeyPass sequences in 47.94% of the wells with the enzyme prelayer were 100% accurate for at least the first 200 sequence positions, as opposed to only 18.08% of those without the prelayer having the same level of accuracy.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A system for reducing crosstalk in a parallel sequencing platform, comprising:
a substrate comprising a plurality of individual reaction environments that include a species of nucleic acid template, and a plurality of spatially localized reactants, wherein the localized reactants minimize the transmission of reaction products to a neighboring reaction environment due to a relative position of the localized reactants in the reaction environment.

2. The system of claim 1, wherein:
the substrate comprises a fiber optic faceplate, where the reaction environments comprise wells etched into a first surface of the fiber optic faceplate.

3. The system of claim 1, wherein:
the spatially localized reactants are disposed on a solid phase substrate.

4. The system of claim 3, wherein:
the solid phase substrate comprises a bead substrate.

5. The system of claim 1, wherein:
the species of nucleic acid template comprises a population of substantially identical copies of a nucleic acid template molecule, wherein the substantially identical copies are disposed on a solid phase substrate.

6. The system of claim 5, wherein:
the solid phase substrate comprises a bead substrate.

7. The system of claim 1, wherein:
the spatially localized regents comprise a first class of reagent and a second class of reagent.

8. The system of claim 7, wherein:
the first class of reagent is selected from the group consisting of pyrophosphatase and apyrase.

9. The system of claim 8 wherein:
the pyrophosphatase removes 90%-99% of pyrophosphate that diffuses into and out of the reaction environment.

10. The system of claim 7, wherein:
the second class of reagent is selected from the group consisting of sulfurylase and luciferase.

11. The system of claim 7, wherein:
the individual reaction environments comprise wells, wherein the first class reagent is localized to a top region of the well and the second class of reagent is localized to a bottom region of the well.

12. The system of claim 11, wherein:
the species of nucleic acid template is disposed on a solid phase substrate localized to a central region of the well, wherein the central region further comprises a plurality of solid phase substrates that are free of reactants and occupy space within the central region.

13. The system of claim 12, wherein:
the nucleic acid template substrate comprises a bead and the reactant-free substrates comprise beads, wherein the nucleic acid template bead and the non-reactant beads are compacted in the central region of the well.

14. The system of claim 13, wherein:
the nucleic acid template bead and the non-reactant beads are compacted by centrifugal force.

15. A method for reducing crosstalk in a parallel sequencing platform, comprising:
providing a substrate comprising a plurality of individual reaction environments that include a species of nucleic acid template, and a plurality of spatially localized reactants; and
exposing the reaction environments to a nucleotide species to produce a detectable signal within one or more of the reaction environments, wherein the localized reactants within the reaction environments minimize the transmission of reaction products to a neighboring reaction environment.

16. The system of claim 15, wherein:
the substrate comprises a fiber optic faceplate, where the reaction environments comprise wells etched into a first surface of the fiber optic faceplate.

17. The method of claim 15, wherein:
the spatially localized reactants are disposed on a solid phase substrate.

18. The method stem of claim 17, wherein:
the solid phase substrate comprises a bead substrate.

19. The system of claim 15, wherein:
the species of nucleic acid template comprises a population of substantially identical copies of a nucleic acid template molecule, wherein the substantially identical copies are disposed on a solid phase substrate.

20. The system of claim 19, wherein:
the solid phase substrate comprises a bead substrate.

21. The method of claim 15, wherein:
the spatially localized regents comprise a first class of reagent and a second class of reagent.

22. The method of claim 21, wherein:
the first class of reagent is selected from the group consisting of pyrophosphatase and apyrase.

23. The method of claim 22 wherein:
the pyrophosphatase removes 90%-99% that diffuses into and out of the reaction environment.

24. The method of claim 21, wherein:
the second class of reagent is selected from the group consisting of sulfurylase and luciferase.

25. The method of claim 21, wherein:
the individual reaction environments comprise wells, wherein the first class reagent is localized to a top region of the well and the second class of reagent is localized to a bottom region of the well.

26. The method of claim 25, wherein:
the nucleic acid template is disposed on a solid phase substrate localized to a central region of the well, wherein the central region further comprises a plurality of solid phase substrates that are free of reactants and occupy space within the central region.

27. The method of claim 26, wherein:
the nucleic acid template substrate comprises a bead and the reactant-free substrates comprises beads, wherein the nucleic acid template bead and the non-reactant beads are compacted in the central region of the well.

28. The method of claim 27, wherein:
the nucleic acid template bead and the non-reactant beads are compacted by centrifugal force.

29. A kit for performing the method of claim 15, comprising:
the substrate comprising a fiber optic faceplate;
the localized reactants comprising a first class of reagent sequestered to a plurality of beads and a second class of reagent sequestered to a plurality of beads; and
a plurality of non-reactant beads.

30. The kit of claim 29, wherein:
the first class of reagent is selected from the group consisting of pyrophosphatase and apyrase.

31. The kit of claim 29, wherein:
the second class of reagent is selected from the group consisting of sulfurylase and luciferase.
